(19) United States
(12) Patent Application Publication
(10) Pub. No.: US 2004/0202693 A1
(43) Pub. Date: Oct. 14, 2004

(54) Pharmaceutical compositions releasing their active agents from a buccal or sublingual location to overcome an absorption window problem

(76) Inventor: Rong-Kun Chang, Rockville, MD (US)

Correspondence Address: M. Elisa Lane, Shire Laboratories, Inc., 1550 East Gude Drive, Rockville, MD, 20850, US

(21) Appl. No.: 10/822,949

(22) Filed: Apr. 13, 2004

Related U.S. Application Data
(60) Provisional application No. 60/462,830, filed on Apr. 14, 2003.

(51) Int. Cl. A61K 38/13; A61K 31/704; A61K 9/48; A61K 31/43

(52) U.S. Cl. 424/428; 514/11; 514/192; 514/34; 514/567; 514/152

(57) ABSTRACT

Disclosed are controlled release dosage forms of pharmaceutical or nutritional agents that are intended for retention in a buccal or sublingual location for administration. The dosage forms are particularly useful for the sustained release administration of drugs that have a limited window of absorption in the gastrointestinal tract and that are minimally, if at all, absorbed mucosally.

### FIELD OF THE INVENTION

The present invention is directed to pharmaceutical dosage forms that are retained in a buccal or sublingual location. Such dosage forms are useful for pharmaceuticals or nutritional substances that exhibit a limited absorption window in the gastrointestinal tract.

### BACKGROUND OF INVENTION

In the market, there are two implantable products for site-specific use in the treatment of periodontal disease. The PerioChip® is a small, orange-brown chip, which is inserted into periodontal pockets. Each PerioChip® contains 2.5 mg of chlorohexidine gluconate in a biodegradable, resorbable matrix. It is recommended that PerioChip® treatment be administered once every three months in pockets that remain at 5 mm or deeper. A second product, Atridox®, is an injectable, resorbable gel, which provides the subgingival controlled-release of 42.5 mg doxycycline for approximately one week. Additionally, there is now available a new oral medication called Periostat®, which delivers 20 mg doxycycline systemically as a collagenase inhibitor used in patients with adult periodontal disease. Most people would prefer to take a pill to the implant. However, Periostat® requires twice daily dosing and raises concerns about patient compliance. Thus, there is great reason to develop a one dose per day sustained-release formulation for doxycycline.

Not all drugs can be absorbed throughout the entire gastrointestinal tract. The examination of drug absorption in different intestinal segment lengths can reveal the presence of an absorption window. Doxycycline is rapidly and almost completely absorbed from the upper portion of the gastrointestinal tract following oral administration in conventional dosage forms. It has been documented that a sustained-release formulation can achieve a degree of sustained effect, but the bioavailability will be significantly compromised. This reduced bioavailability is confirmation of an absorption window. Trospium chloride is poorly absorbed after oral administration; its bioavailability is approximately 10%. An enteric-coated trospium chloride formulation results in a significant decrease of bioavailability. After rectal administration, there is almost no absorption at all. The decrease of trospium chloride bioavailability along the gastrointestinal tract suggests that its absorption is limited to the upper small intestine.

Other than doxycycline and trospium chloride, there are many drugs (e.g., clonazepam, cyclosporin, ampicillin, amoxicillin, riboflavin, levadopa, talinolol, furosemide, cefixime) that have the absorption window problem. For such drugs, the transit time through the gastrointestinal tract often limits the amount of drug available for absorption at its most efficient absorption site. This often results in low bioavailability. This is particularly true when the absorption site is high in the gastrointestinal tract, for instance the upper small intestine. To design a sustained-release oral dosage form for drugs with an absorption window problem is extremely difficult because of the loss of bioavailability and lack of sustained effect.

To overcome these problems, the gastric retentive dosage forms based on various mechanisms, such as with bioadhesive, buoyancy, size, shape, and chemicals with the ability to retard gastrointestinal motility, have been investigated extensively. However, to date, no reliable and acceptable systems are available to achieve gastric retention.

### DESCRIPTION OF INVENTION

The present invention is directed to a dosage form, which is retained in a buccal or sublingual location via a bioadhesive mechanism or a holding device, and which provides sustained release of a pharmaceutical or nutriceutical that has a limited absorption window in the gastrointestinal tract and is minimally, if at all, mucosally absorbed. Two such drugs are doxycycline or its salts and trospium chloride, although the present invention is contemplated to apply to any drug that has an absorption window limited to the upper gastrointestinal tract (i.e. upper and mid-small intestine, or less than about 6 hours after ingestion).

Also, the present invention provides a method of administering to a patient a pharmaceutically active agent that has an absorption window of less than 6 hours in a sustained release fashion, wherein a sustained release matrix dosage form is placed into the buccal or sublingual cavity of the patient for a certain period of time, e.g. up to 6 hours.

The invention provides the notion of retaining a sustained-release dosage form in a buccal/sublingual location, which will gradually release the drug for systemic absorption. This approach is quite different from conventional buccal tablets, which provide systemic drug delivery via the oral mucosal route.

The dosage form is placed and held in the mouth, as with other buccal dosage forms, for as long as 6 hours. The active pharmaceutical is one that does not, and is not intended to, absorb through the oral mucosa to any appreciable extent. Not only would bioavailability increase with such dosage forms, but also the dosage form can be effective as a sustained release of a pharmaceutical that otherwise could not have a sustained release because of the limited absorption window. Thus, the present invention overcomes the problems of low bioavailability and lack of sustained effect inherent with some pharmaceuticals.

The dosage form of the present invention is preferably a sustained release type of formulation. For a sustained-release matrix, utilization of a hydrophilic matrix as a means to control drug release was disclosed in U.S. Pat. No. 3,065,143, which is hereby incorporated by reference. Sodium carboxymethylcellulose, methylcellulose, hydroxypropylcellulose, hydroxyethyl cellulose, polyethylene oxide, polyvinyl pyrrolidone, polyvinyl acetate, carboxyl polymethylene, alginic acid, gelatin, and nature gums can be used as matrix materials. The matrix may be tableted by direct compression of the blend of active ingredient(s) and certain hydrophilic matrix materials or from a wet granulation containing the drug, hydrophilic matrix materials, and other compression aids.

Compressed hydrophilic matrices have an effect on formulation and processing variables and on drug-release behavior. Therefore, preferably, the matrix building material with fast polymer hydration capability is the best choice to use in a hydrophilic matrix tablet formulation. An inadequate polymer hydration rate may cause premature diffusion of the drug and disintegration of the tablet owing to fast penetration of water. This is particularly true for formulation of water-soluble drugs and excipients.

The amount of hydrophilic polymer in tablet formulations has been reported to have a marked influence on the disintegration time and dissolution of a tablet. The disintegration time was extended, however, as polymer content increased. The release rate of drug was decreased when the proportion of polymer was increased, but differed quantitatively with different drugs and different matrix-building materials. Slower hydration polymers can be used at higher concentration level to accelerate gel formation or reserved for water-insoluble drugs.

Generally, reduced particle size of the hydrophilic polymer ensures rapid hydration and gel formation, leading to a good controlled release. The impact of polymer particle size on the release rate is formulation dependent, but may be obscured in some cases. The particle size of a drug, within a normal size, may not significantly influence the drug release from the matrix tablet. Extremes of drug particle size may affect release rate of the drug.

Viscosity characteristics of the polymers are of great importance in determining the final release properties of the matrix tablet. Generally, the drug release rate is slower for a higher viscosity grade polymer.

Commonly, water-soluble excipients in the matrix tablet can increase drug release. However, addition of water-soluble materials may achieve a slower rate by increasing viscosity of the gel through interaction with hydrophilic polymers or by competition with matrix material for water. When water-insoluble nonswellable excipients or drugs is used in the matrix system, stress cracks can occur upon immersion in water because of the combination of swelling and nonswelling components on the tablet surface.

For some hydrophilic matrix building materials, pH may affect the viscosity of the gel that forms on the tablet surface and its subsequent rate of hydration. Under acidic conditions, carboxypolymethylene and sodium carboxymethyl cellulose have little or no retarding effect on drug release rate. Gelatin forms gels of higher viscosity in acidic media and is more effective in retarding drug release as compared to a basic media.

Compression force, tablet size, and tablet shape can significantly influence drug-release kinetics. The drug can be incorporated into fat-wax granulations by spray congealing in air, blend congealing in an aqueous media with or without the aid of surfactants, and spray-drying techniques. In the bulk congealing method, a suspension of drug and melted fat-max is allowed to solidify and is then comminuted for sustained-release granulations. The mixture of active ingredients, wax materials, and fillers also can be converted into granules by compacting with a roller compactor, heating in a suitable mixer such as a fluidized-bed and steam-jacketed blender, or granulating with a solution of waxy material or other binders.

Fat-wax granulations containing drug obtained from all of the above processes may be compressed into a tablet with sustained-release properties. The drug embedded into a melt of fats and waxes is released by leaching and/or hydrolysis as well as dissolution of fats under influence of enzymes and pH change in the gastrointestinal tract. In general, the primary constituents of a fat-wax matrix are fatty acids, fatty alcohol, and/or fatty esters. Fatty acids are more soluble in an alkaline rather than an acidic medium. Fatty esters are more susceptible to alkaline catalyzed hydrolysis than to acid catalyzed hydrolysis. The surface erosion of a fat-wax matrix depends upon the nature and percent of fat-wax and extenders in the matrix.

Other factors, such as drug particle size and drug concentration, affect release of the drug from the matrix system. The addition of surfactants to the formulation can also influence both the drug-release rate and the proportion of total drug that can be incorporated into a matrix. Polyethylene glycol, ethylcellulose, polyethylene, sugars, and sugar alcohols were added to modify the drug release pattern.

Sustained-release tablets based upon an inert compressed plastic matrix were first introduced in 1960 and have been used extensively clinically. Release is usually delayed because the dissolved drug has to diffuse through a capillary network between the compacted polymer particles. Matrix formulations are well known. Commonly used plastic matrix materials are polyvinyl chloride, polyethylene, vinyl acetate/vinyl chloride copolymer, vinylidene chloride/acrylonitrile copolymer, acrylate methylmethacrylate copolymer, ethyl cellulose, cellulose acetate, and polystyrene.

Plastic matrix tablets, in which the active ingredient is embedded in a tablet with a coherent and porous skeletal structure, can be easily prepared by direct compression of drug with plastic material(s), provided the plastic material can be comminuted or granulated to the desired particle size to facilitate mixing with drug particle. In order to granulate for compression into tablets, the embedding process may be accomplished by: (a) the solid drug and the plastic powder can be mixed and kneaded with a solution of the same plastic material or other binding agents in an organic solvent and then granulated; (b) the drug can be dissolved in the plastic by using an organic solvent and granulated upon evaporation of the solvent; using latex/pseudolatex as granulating fluid to granulate the drug and plastic masses.

Drug release from the inert plastic matrices is affected by varying formulation factors, such as the matrix material, amount of drug incorporated in the matrix, drug solubility in the dissolution media and in the matrix and matrix additives. Since the mechanism of controlling drug release in the plastic matrix is the pore structure of the matrix, any formulation factors affecting the release of a drug from the matrix may be a consequence of their primary effect on apparent porosities and tortuosities of the matrices. These release factors can be summarized as follows:

1. The release rate increases as the solubility of the drug increases; the release rate increases as the drug concentration increases.

2. It is possible to modify the release rate by inclusion of hydrophilic or hydrophobic additives to the matrix. The release of a sparingly soluble substance can be increased by the addition of physiologically inert but readily soluble material such as polyethylene glycol, sugars, sugar alcohols, electrolytes, and urea. The decrease in the release rate on the addition of hydrophobic substance may be due to decreased wettability of the matrix.

3. The release rate increased as the particle size of the matrix material increased and as the particle size of the drug decreased.

4. Increasing compaction pressure up to the full consolidation point tends to decrease the pore formed among the polymer particles, resulting in a slower drug-release rate.

Additionally, a layer tablet approach, which consists of one fast dissolving layer and one adhesive sustained release layer, can be used to fabricate the buccal system. Such dosage forms and their preparation are disclosed in Gunsel and Dusel, Chapter 5, “Compression-coated and layer tablets”, in Pharmaceutical Dosage Forms:Tablets, Second Edition, Volume 1, Edited by H. A. Lieberman, L. Lachman, and J. B. Schwartz, Marcel Dekker, Inc. New York and Basel (1990), which is hereby incorporated herein by reference. This publication gives a review of techniques well known in the art of making layered tablets by compression coating, tablet inlaying and sandwich-type layering.

The dosage forms of the present invention can be tablets or discs. Discs can be fabricated by compression, molding, extrusion or laminating. No matter what method is used to prepare them, discs are generally a cylindrical-shaped device. However, other shapes such as rectangular can be fabricated.

For mucoadhesives, any of the commonly used substances, as disclosed in Shojaei et al., “Systemic drug delivery via the buccal mucosal route,” Pharmaceutical Technology, June 2001, pp.70-81, incorporated herein by reference, may be used with the present invention. These include synthetic polymers such as monomeric a cyanolacrylate, polyacrylic acid, hydroxypropyl methylcellulose, and polymethacrylate derivatives as well as naturally occurring polymers such as hyaluronic acid and chitosan. Other non-limiting examples include: hydropropyl cellulose and Carbopol, alone or in combination; poly(vinyl pyrrolidone; sodium carboxymethyl cellulose; hydroxyethyl cellulose; poly(vinyl alcohol); poly(isobutylene); poly(isoprene); xanthum gum; locust bean gum; polycarbophil; and poly(acrylic acid-co-poly ethylene glycol). See further Table II of the Shojaei publication.

The holding device, if needed, can be a plastic holder with string and the tablet can be inserted into the plastic holder and the string can be attached to the teeth to retain the dosage form in the oral cavity. The holding device also can be a dental polymeric strip containing drugs, which can be attached to teeth.

The present invention is exemplified in the following examples, it being understood that the invention is not thereby limited.

### EXAMPLES

### Example 1

A sustained-release tablet formulation with a mucoadhesive material was investigated. The formula contains the following: Carbopol 971 (18.75%), Xylitab® (31.25%), aspartame (1.25%), lemonade flavoring agent (1.25%), silicified microcrystalline cellulose (19.375%), magnesium stearate (1.25%) and doxycycline monohydrate drug substance. Percentages are by weight, unless otherwise noted. The powder was blended and granulated using isopropyl alcohol as a granulating fluid. The dried granulation was blended with magnesium stearate and compressed into tablets.

The bitter taste of doxycycline monohydrate was successfully masked by using the flavoring and sweetening agents. The tablet was able to adhere to the mucosal lining in a location within the mouth for a long period time.

The dissolution data are as follows: 16% in 0.5 hour, 25% in 1 hour, 38% in 2 hour, 43% in 2.5 hour, 46% in 3 hour, 49% in 4 hour, and 51% in 5 hour. Thus, the formulation gave a sustained-release profile. The dissolution profile can be easily modified, for instance as described in this application, to achieve the desired dissolution characteristics.

What is claimed is:

1. A sustained release pharmaceutical dosage form, which is held in a buccal or sublingual location, comprising a pharmaceutically or nutritionally active agent that exhibits absorption window of less than 6 hours in the gastrointestinal tract, in a sustained release matrix formulation, whereby the active agent is released and swallowed gradually over an extended time period and absorbed systemically in the gastrointestinal tract.

2. The dosage form of claim 1, wherein the matrix is composed of a hydrophilic polymer matrix, a fat-wax matrix, or an inert plastic matrix.

3. The dosage form of claim 1, which is a layered tablet.

4. The dosage form of claim 1, wherein one surface of the dosage form contains a mucoadhesive, which will function to hold the dosage for in place in the buccal or sublingual location.

5. The dosage form of claim 1, wherein the matrix formulation is held in the buccal or sublingual location by a holding device.

6. The dosage form of claim 1, wherein the pharmaceutically or nutritionally active agent is one that is not absorbed through the oral mucosa to a substantial extent.

7. The dosage form of claim 1, wherein the active agent is doxycycline, trospium chloride, clonazepam, ampicillin, amoxicillin, riboflavin, levadopa, talinolol, furosemide, cefixime or cyclosporin.

8. A method of administering to a patient a pharmaceutically or nutritionally active agent that has an absorption window of less than 6 hours in a sustained release fashion, comprising placing a sustained release matrix dosage form into the buccal or sublingual cavity of the patient.

9. The method of claim 8, wherein the dosage form matrix is composed of a hydrophilic polymer matrix, a fat-wax matrix, or an inert plastic matrix.

10. The method of claim 8, wherein the dosage form is a layered tablet.

11. The method of claim 8, wherein one surface of the dosage form contains a mucoadhesive, which will function to hold the dosage for in place in the buccal or sublingual location.

12. The method of claim 8, wherein the matrix formulation is held in the buccal or sublingual location by a holding device.

13. The method of claim 8, wherein the active agent is doxycycline, trospium chloride, clonazepam, ampicillin, amoxicillin, riboflavin, levadopa, talinolol, furosemide, cefixime, or cyclosporin.

14. A process for preparing the dosage form of claim 1, comprising combining a pharmaceutically or nutritionally active agent with matrix materials and fabricating into a tablet or disc.

15. The process of claim 14, further comprising applying a mucoadhesive to one surface of the tablet or disc.
